(19) United States
(12) Patent Application Publication
(10) Pub. No.: US 2016/0287416 A1
(43) Pub. Date: Oct. 6, 2016

(54) METHOD TO CREATE MICROPATTERNS ON AN INSIDE SURFACE OF A STENT

(71) Applicant: BOSTON SCIENTIFIC SCIMED, INC., Maple Grove, MN (US)

(72) Inventor: Jan Weber, Maastricht (NL)

(21) Appl. No.: 15/085,287

(22) Filed: Mar. 30, 2016

Related U.S. Application Data
(60) Provisional application No. 62/141,585, filed on Apr. 1, 2015.

(51) Int. Cl. A61F 2/82

(52) U.S. Cl. CPC A61F 2/82; A61F 2230/0069; A61F 2240/001; A61F 2250/0067

(57) ABSTRACT

A method for manufacturing a tubular medical device having a micropatterned inner surface is disclosed. A glass tube having an optical mask on an outer surface thereof may be placed within a lumen of a tubular medical device, wherein the optical mask forms a pattern of shapes. An ultraviolet light source may be advanced within a lumen of the glass tube. The inner surface, including a photoresist coating, of the tubular medical device may be illuminated with ultraviolet light through the glass tube. The optical mask may block ultraviolet light from passing through portions of the glass tube. The inner surface of the tubular medical device may be etched to create a plurality of protrusions.

### CROSS-REFERENCE TO RELATED APPLICATIONS

This application claims priority under 35 U.S.C. §119 to U.S. Provisional Application Ser. No. 62/141,585, filed Apr. 1, 2015, the entirety of which is incorporated herein by reference.

### TECHNICAL FIELD

The present disclosure pertains to medical devices, and methods for manufacturing medical devices. More particularly, the present disclosure pertains to micropatterns on an inside surface of a stent and methods of creating micropatterns on an inside surface of a stent.

### BACKGROUND

Implantable stents are devices that are placed in a body structure, such as a blood vessel or body cavity, to provide support and to maintain the structure open. These devices are manufactured by any one of a variety of different manufacturing methods and may be used according to any one of a variety of methods.

### SUMMARY

This disclosure provides design, material, manufacturing method, and use alternatives for medical devices. An example medical device may include a stent. An example method of manufacturing a curved medical device stent comprises:

placing a glass tube having an optical mask on an outer surface thereof adjacent to a concave surface of a curved medical device, wherein the optical mask forms a pattern and the concave surface of the curved medical device having a light sensitive photoresist layer disposed thereon;

advancing an ultraviolet light source within a lumen of the glass tube;

illuminating the concave surface of the curved medical device with ultraviolet light through the glass tube, wherein the optical mask blocks ultraviolet light from passing through portions of the glass tube; and

etching the concave surface of the curved medical device to create a plurality of protrusions.

Alternatively or additionally to any of the embodiments above, the pattern is formed of a plurality of shapes, the shapes having a length in the range of 1 to 100 micrometers.

Alternatively or additionally to any of the embodiments above, the plurality of protrusion have a cross-sectional shape that is a negative image of the shapes of the pattern.

Alternatively or additionally to any of the embodiments above, wherein illuminating the concave surface of the curved medical device with ultraviolet light through the glass tube comprises rotating and/or translating the ultraviolet light source along a longitudinal axis of the glass tube.

Alternatively or additionally to any of the embodiments above, wherein placing a glass tube having an optical mask on an outer surface thereof adjacent to a concave surface of a curved medical device further comprises applying a force to the curved medical device to bring the concave surface into contact with the outer surface of the glass tube.

Alternatively or additionally to any of the embodiments above, the pattern extends over a portion of the outer surface of the glass tube.

Alternatively or additionally to any of the embodiments above, the pattern extends over the entire outer surface of the glass tube.

Alternatively or additionally to any of the embodiments above, a height of the plurality of protrusions is determined by a processing time of the etching of the concave surface.

An example endoluminal implant comprises:

an elongated tubular body having an inner surface and an outer surface;

wherein the inner surface comprises a micropatterned surface including a plurality of protrusions, the plurality of protrusions formed as a monolithic structure with elongated tubular body.

Alternatively or additionally to any of the embodiments above, the plurality of protrusions each have a length in the range of 1 to 100 micrometers.

Alternatively or additionally to any of the embodiments above, the plurality of protrusions each have a height in the range of 1 to 100 micrometers.

Alternatively or additionally to any of the embodiments above, the micropatterned surface extends over a portion of the inner surface of the elongated tubular body.

Alternatively or additionally to any of the embodiments above, the micropatterned surface extends over the entire inner surface of the elongated tubular body.

Alternatively or additionally to any of the embodiments above, the endoluminal implant further comprising a coating disposed over the micropatterned surface, the coating selected to stimulate cell growth.

Alternatively or additionally to any of the embodiments above, the plurality of protrusions each have cross-sectional shape selected to promote cell growth.

An example method for manufacturing a curved medical device comprises:

placing a glass tube having an optical mask on an outer surface thereof adjacent to a concave surface of a curved medical device, wherein the optical mask forms a pattern and the concave surface of the curved medical device having a light sensitive photoresist layer disposed thereon;

advancing an ultraviolet light source within a lumen of the glass tube;

illuminating the concave surface of the curved medical device with ultraviolet light through the glass tube, wherein the optical mask blocks ultraviolet light from passing through portions of the glass tube; and

etching the concave surface of the curved medical device to create a plurality of protrusions.

Alternatively or additionally to any of the embodiments above, the pattern is formed of a plurality of shapes, the shapes having a length in the range of 1 to 100 micrometers.

Alternatively or additionally to any of the embodiments above, the plurality of protrusion have a cross-sectional shape that is a negative image of the shapes of the pattern.

Alternatively or additionally to any of the embodiments above, wherein illuminating the concave surface of the curved medical device with ultraviolet light through the glass tube comprises rotating and/or translating the ultraviolet light source along a longitudinal axis of the glass tube.

Alternatively or additionally to any of the embodiments above, wherein placing a glass tube having an optical mask forming a pattern adjacent to a concave surface of a curved medical device further comprises applying a force to the curved medical device to bring the concave surface into contact with the outer surface of the glass tube.

Alternatively or additionally to any of the embodiments above, the pattern extends over a portion of the outer surface of the glass tube.

Alternatively or additionally to any of the embodiments above, pattern extends over the entire outer surface of the glass tube.

Alternatively or additionally to any of the embodiments above, a height of the plurality of protrusions is determined by a processing time of the etching of the concave surface.

An example method for manufacturing a tubular medical device comprises:

placing a glass tube having an optical mask on an outer surface thereof within a lumen of a tubular medical device, wherein the optical mask forms a pattern of shapes;

advancing an ultraviolet light source within a lumen of the glass tube;

illuminating an inner surface including a photoresist coating of the tubular medical device with ultraviolet light through the glass tube, wherein the optical mask blocks ultraviolet light from passing through portions of the glass tube; and

etching the inner surface of the tubular medical device to create a plurality of protrusions.

Alternatively or additionally to any of the embodiments above, further comprising applying a force to an outer surface of the tubular medical device to bring the inner surface of the tubular medical device into contact with the outer surface of the glass tube prior to illuminating the inner surface of the tubular medical device.

Alternatively or additionally to any of the embodiments above, the shapes in the pattern of shapes having a length in the range of 1 to 100 micrometers.

Alternatively or additionally to any of the embodiments above, wherein illuminating an inner surface of the tubular medical device comprises rotating and/or translating the ultraviolet light source along a longitudinal axis of the glass tube.

Alternatively or additionally to any of the embodiments above, further comprising applying a coating over the plurality of protrusions.

An example endoluminal implant comprises:

an elongated tubular body having an inner surface and an outer surface;

wherein the inner surface comprises a micropatterned surface including a plurality of protrusions, the plurality of protrusions formed as a monolithic structure with elongated tubular body.

Alternatively or additionally to any of the embodiments above, the plurality of protrusions each have a length in the range of 1 to 100 micrometers.

Alternatively or additionally to any of the embodiments above, the plurality of protrusions each have a height in the range of 1 to 100 micrometers.

Alternatively or additionally to any of the embodiments above, the micropatterned surface extends over a portion of the inner surface of the elongated tubular body.

Alternatively or additionally to any of the embodiments above, the micropatterned surface extends over the entire inner surface of the elongated tubular body.

Alternatively or additionally to any of the embodiments above, the endoluminal implant further comprising a coating disposed over the micropatterned surface, the coating selected to stimulate cell growth.

Alternatively or additionally to any of the embodiments above, wherein the plurality of protrusions each have cross-sectional shape selected to promote cell growth.

An illustrative method for manufacturing a curved medical device may comprise placing a glass tube having an optical mask on an outer surface thereof adjacent to a concave surface of a curved medical device. The optical mask may form a pattern and the concave surface of the curved medical device may have a light sensitive photoresist layer disposed thereon. An ultraviolet light source may be advanced within a lumen of the glass tube. The concave surface of the curved medical device may then be illuminated with ultraviolet light through the glass tube. The optical mask may block ultraviolet light from passing through portions of the glass tube. The concave surface of the curved medical device may be etched to create a plurality of protrusions.

Another illustrative method for manufacturing a tubular medical device may comprise placing a glass tube having an optical mask on an outer surface thereof within a lumen of a tubular medical device. The optical mask may form a pattern of shapes. An ultraviolet light source may be advanced within a lumen of the glass tube. An inner surface of the tubular medical device including a photoresist coating may be illuminated with ultraviolet light through the glass tube. The optical mask may block ultraviolet light from passing through portions of the glass tube. The inner surface of the tubular medical device may be etched to create a plurality of protrusions.

An illustrative endoluminal implant may comprise an elongated tubular body having an inner surface and an outer surface. The inner surface may comprise a micropatterned surface including a plurality of protrusions. The plurality of protrusions may be formed as a monolithic structure with elongated tubular body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 is a side view of an illustrative stent.

FIG. 2 is a cross-sectional view of the illustrative stent of FIG. 1.

FIGS. 3A-3C are illustrative micropatterns.

FIGS. 4-8 are more illustrative micropatterns.

FIG. 9 is an illustrative assembly for forming micropatterns on an inside surface of a stent.

FIG. 10 is a partial cross-sectional view of the assembly of FIG. 9.

FIG. 11 is a partial cross-sectional view of another illustrative micropatterned surface.

FIG. 12 is a partial cross-sectional view of another illustrative micropatterned surface.

### DETAILED DESCRIPTION

Studies have shown that certain micropatterns on medical devices have an effect on the endothelial cell rate of metallic and polymeric substrates. In some instances, in-vitro tests have demonstrated a difference as high as a factor of five between standard smooth surfaces and micropatterned surfaces. It is contemplated that bare metal stents, polymeric stents, biostable stents, and/or biogradable stents may benefit from including micropatterns. In some instances, it may be desirable to provide micropatterns on an inside, or luminal, surface of the stent to promote or inhibit cell growth within the lumen of the stent, as desired. For example, in the case of magnesium bioabsorbable stents, a fast endothelization will give the additional benefit of reducing the corrosion rate by not exposing the stent surface to the blood stream.

While the embodiments disclosed herein are discussed with reference to stents, it is contemplated that the patterns and techniques described herein may be used in other devices, such as, but not limited to, grafts, stent-grafts, vena cava filters, expandable frameworks, etc. It is further contemplated that the devices and methods described herein may be used and sized for use in locations such as, but not limited to: bodily tissue, bodily organs, vascular lumens, non-vascular lumens and combinations thereof, such as, but not limited to, in the coronary or peripheral vasculature, trachea, esophagus, bronchi, colon, small intestine, biliary tract, urinary tract, prostate, brain, stomach and the like.

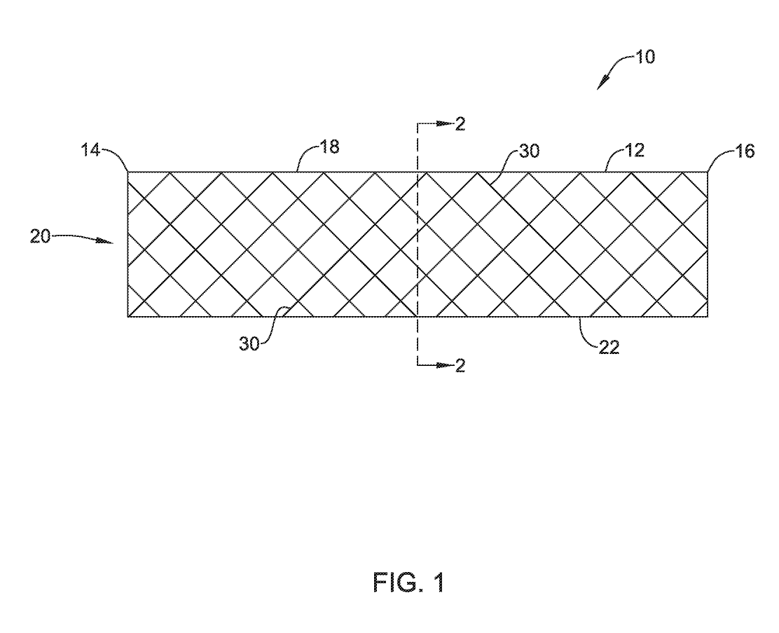

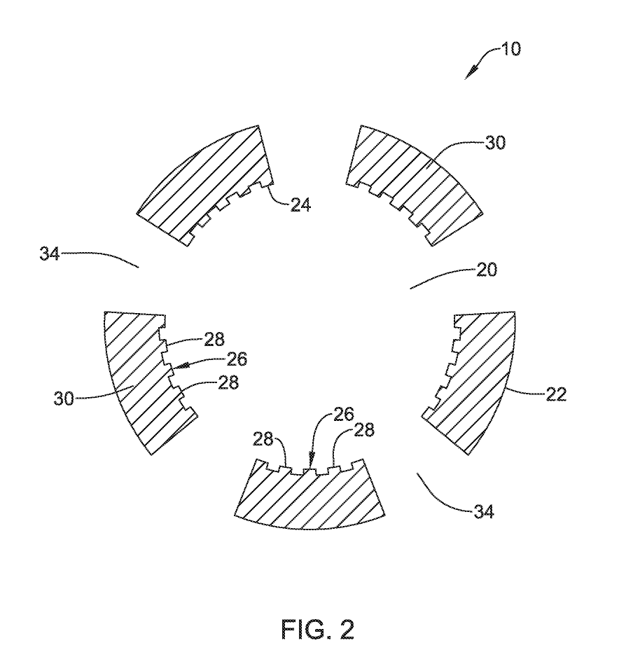

FIG. 1 shows side view of an illustrative endoluminal implant 10, such as, but not limited to, a stent. FIG. 2 illustrates a cross-sectional view of the stent 10 of FIG. 1, taken at line 2-2. In some instances, the stent 10 may be formed from an elongated tubular member 12. While the stent 10 is described as generally tubular, it is contemplated that the stent 10 may take any cross-sectional shape desired. The stent 10 may have a first, or proximal, end 14, a second, or distal, end 16, and an intermediate region 18 disposed between the first end 14 and the second end 16. The stent 10 may include an outer surface 22 and an inner surface 24 (see FIG. 2) defining a lumen 20 extending from a first opening adjacent the first end 14 to a second opening adjacent to the second end 16 to allow for the passage of food, fluids, etc.

The stent 10 may be expandable from a first collapsed configuration (not explicitly shown) to a second expanded configuration. The expandable stent 10 can be self-expanding, balloon expandable, or a combination thereof. The stent 10 may be structured to extend across a stricture and to apply a radially outward pressure to the stricture in a lumen to open the lumen and allow for the passage of foods, fluids, blood, air, etc. In some embodiments, the stent 10 may have a constant diameter, tapers, flares and/or other changes in diameter in the body 12, intermediate region 18, and/or at an end 14, 16, as desired.

The stent 10 may have a cellular structure, fabricated from a number of filaments or struts 30. It is contemplated that the stent 10 may be manufactured in any manner that results in a smooth inner surface 24. For example, the stent 10 may be a laser cut tubular member, such as the EPICTM stents made by Boston Scientific, Corporation. A laser cut tubular member may have an open and/or closed cell geometry including one or more interconnected filaments.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, nitinol and Elgiloy®. Depending on the material selected for construction, the stent 10 may be self-expanding or require an external force to expand the stent 10. In some embodiments, fibers may be used to make the stent 10, which may be cored fibers, for example, having an outer shell made of nitinol having a platinum core. It is further contemplated that the stent 10 may be formed from polymers including, but not limited to, polyethylene terephthalate (PET), polylactic acid, or poly(trimethylene carbonate).

In some instances, the inner surface 24 of the stent 10 may include a textured, or micropatterned surface 26. For example, as shown in FIG. 2, the micropatterned surface 26 of the stent 10 may include a plurality of protrusions 28. The protrusions 28 may be formed as a monolithic structure with the tubular body 12. For example, as will be discussed in more detail below, the protrusions 28 may be formed by removing portions of the stent body 12 and/or struts 30 such that the protrusions 28 are seamless with the stent body 12 and/or struts 30. In one example, a laser cut stent 10 may be formed from an extruded or drawn tube. Portions of the inner surface 24 may be mechanically, chemically, or optically removed to form a monolithic stent 10 having a seamless micropatterned surface 26 with protrusions 28 along the inner surface 24. In another example, portions of an individual filament or strut 30 may be mechanically, chemically, or optically removed to form a seamless monolithic strut 30 having a micropatterned surface 26 with protrusions 28.

In some embodiments, the micropatterned surface 26 may extend from the first end 14 to the second end 16 or over substantially the entire inner surface 24 of the stent 10. In other embodiments, the micropatterned surface 26 may extend over a portion of the inner surface 24 of the stent 10. For example, the micropatterned surface 26 may extend from the proximal end 14 to a point proximal of the distal end 16. Alternatively, or additionally, the micropatterned surface 26 may extend from the distal end 16 to a point distal of the proximal end 14. Other configurations in which the micropatterned surface 26 covers a portion of the inner surface 24 are also contemplated. In some instances, the micropatterned surface 26 may be formed in one or more discrete sections along a length or circumference of the inner surface 24 of the stent 10. For example, the micropatterned surface 26 may extend in intermittent rings around the inner surface 24 or in intermittent stripes along a length thereof. These are just examples. It is contemplated that discrete sections of the micropatterned surface 26 may be separated by smooth sections, or sections that do not have protrusions 28 extending therefrom.

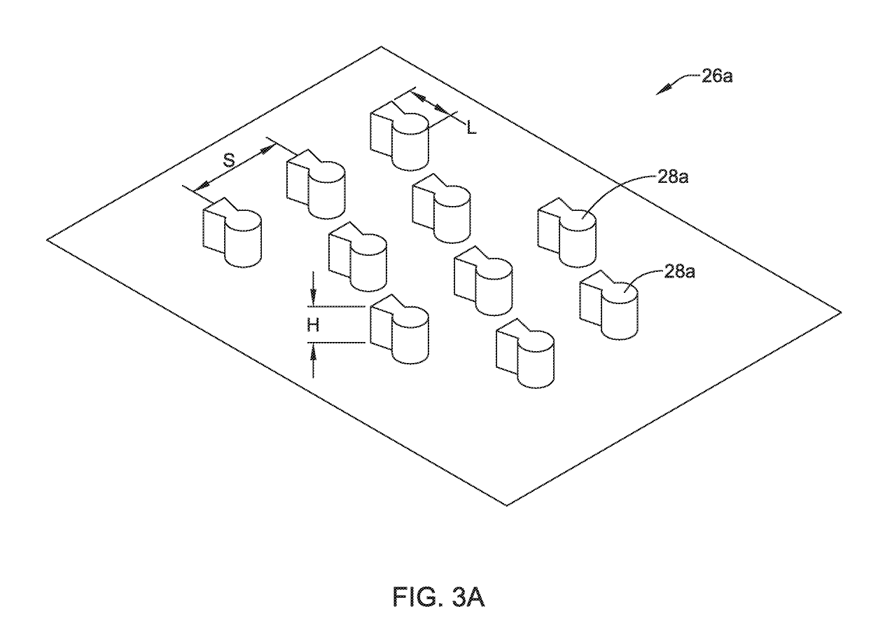

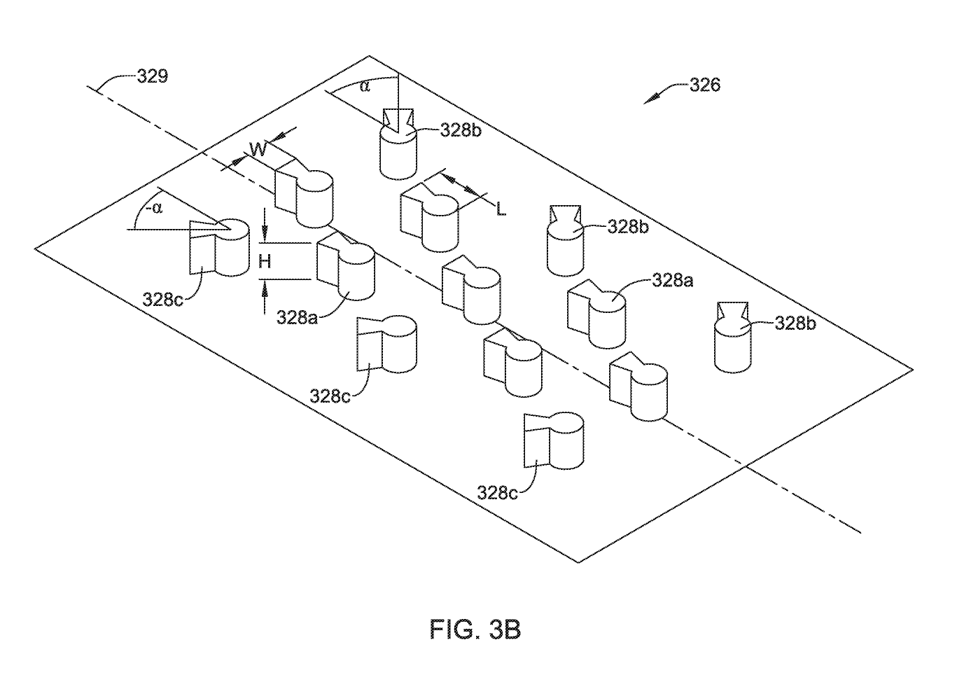

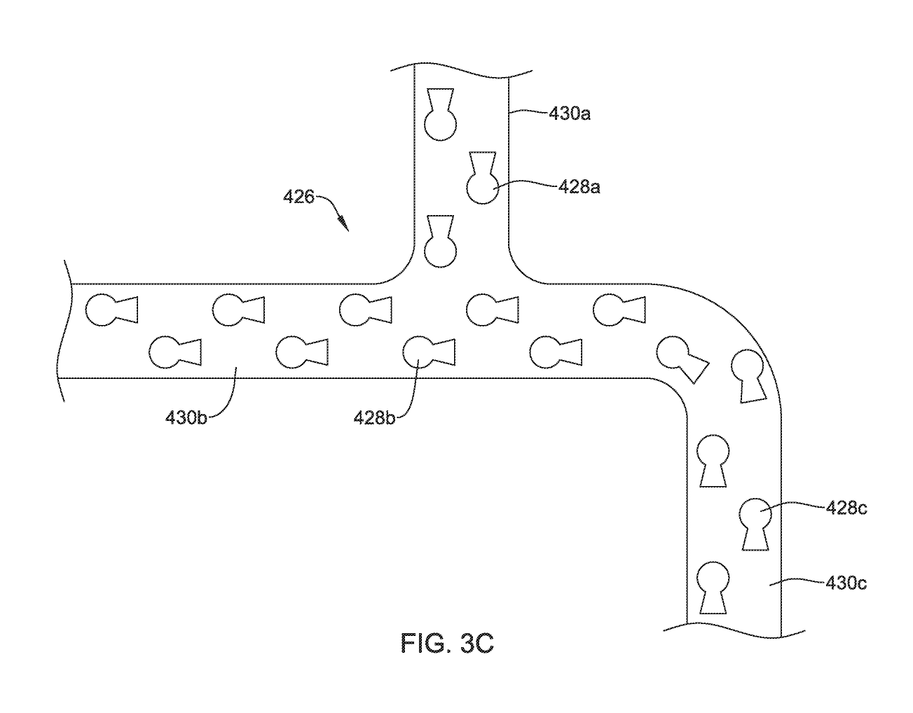

The size, shape, angular orientation, and/or distribution of the protrusions 28 may be selected based on the desired endothelial response once the stent 10 is placed in the body. For example, certain shapes may inhibit cell growth while other shapes may encourage rapid cell growth. It is further contemplated that the size and shape of the protrusions 28 may be selected to encourage a particular type of cell growth. FIGS. 3-8 illustrate some potential shapes and patterns of the protrusions 28 forming the micropatterned surface 26. These are just examples. It is contemplated that the micropatterned surface 26 may include protrusions 28 having any cross-sectional shape desired, such as, but not limited to, circular, cylindrical oval, columnar, square, rectangular, polygonal, triangular, or combinations thereof. It is further contemplated that the size of the cross-sectional size and/or shape may vary along the height of the protrusion 28. For example, the protrusions 28 may be conical, hemispherical, pyramidal, etc. In some embodiments, the protrusions 28 of the micropatterned surface 26 may all have the same shape. In other embodiments, the protrusions 28 of the micropatterned surface 26 may include two or more different shapes depending on the desired response. For example, a portion of the micropatterned surface 26 may include protrusions 28 which encourage cell growth, while another portion of the micropatterned surface 26 may include protrusions 28 which inhibit cell growth.

FIG. 3A illustrates an enlarged and flattened view of illustrative protrusions 28a forming a micropatterned surface 26a. In some instances, the protrusions 28a may be a combination of basic shapes to form a more complicated shape. For example, in FIG. 3A a circle and a triangle have been combined to form a protrusion 28a having a keyhole shaped cross-section. Each protrusion 28a of the micropatterned surface 26a may have a first dimension, or length L, and a second dimension, or height H. The length L of the protrusion 28a may be defined as the greatest distance between two opposite sides of the protrusions 28a. The length L of the keyhole shaped protrusion 28a is the distance between the base of the triangular portion and the edge of the circular portion. The first dimension may be in the range of about 1 micrometer (μm) to 100 μm and the second dimension may be in the range of about 1 μm to 100 μm. It is contemplated that each protrusion 28a may be spaced apart from an adjacent protrusion by a distance S. The distance S may be selected based on the desired application. In some instances, some protrusions 28a may be staggered from the protrusions 28a in adjacent rows, as shown in FIG. 3. In other instances, the protrusions 28a may be uniformly distributed in an array. The protrusions 28a may be arranged in other patterns or configurations as desired, such as, but not limited to, helical rows or a random orientation.

FIG. 3B illustrates an enlarged and flattened view of illustrative protrusions 328a, 328b, 328c (collectively 328) forming a micropatterned surface 326. In FIG. 3B, the protrusions 328 have a keyhole shaped cross-section. Each protrusion 328 of the micropatterned surface 326 may have a first dimension, or length L, a second dimension, or height H, and a third dimension, or width W. The first dimension may be in the range of about 1 μm to 100 μm and the second dimension may be in the range of about 1 μm to 100 μm. In some instances, one or more of the protrusions 328b, 328c may be positioned such that the first dimension and/or third dimension is oriented at a non-zero angle α, −α to a longitudinal axis 329 of the micropatterned surface 326. In other instances, one or more of the protrusions 328a may be oriented such that the first dimension and/or third dimension is oriented parallel to a longitudinal axis. The protrusions 328 may be arranged such that some of the protrusions 328b, 328c are at a non-zero angle α, −α to the longitudinal axis 329 while other protrusions 328a are parallel to the longitudinal axis 329. It is contemplated that the protrusions 328 may be positioned such that a mirror image is generated about the longitudinal axis 329. For example some protrusions 328b on a first side of the longitudinal axis 329 may be oriented at a first non-zero angle α to the longitudinal axis 329 while some protrusions 328c on a second side of the longitudinal axis may be oriented at a second non-zero angle −α, which may be equal in magnitude to the first non-zero angle α. This is just an example. It is contemplated that the protrusions 328 may be arranged in any configuration desired. In some embodiments, the protrusions 328 may be arranged, or angled, according to a distance from an edge of the stent strut, such as strut 30. For example, an angle of the protrusions 328 relative to the longitudinal axis 329, such as, but not limited to angles α, −α, may vary or change across a width of the strut. In some embodiments, a stent strut, such as strut 30, may be a width in the range of 50 to 100 μm. In some instances, the strut may include one, two, ten, twenty, or more protrusions 328 positioned across a width of a strut. This is just an example. The size, spacing, and/or quantity of the protrusions 328 may be varied, as desired, to achieve the desired effect.

FIG. 3C illustrates an enlarged and flattened view of a portion of an illustrative stent, such as stent 30, including a plurality of struts 430a, 430b, 430c (collectively 430). The struts 430 may include a plurality of protrusions 428a, 428b, 428c (collectively 428) forming a micropatterned surface 426 on a surface of the struts 430. In some instances, the micropatterned surface 426 may be on an inner surface of the struts 430, while in other instances, the micropatterned surface 426 may be on an outer or side surface of the struts 430. It is contemplated that the effect of the micropatterned surface 426 on endothelial cell rate may also be is influenced by the blood-flow direction. As each stent strut 430a, 430b, 430c is oriented differently in the vessel, every strut 430 could have its own angular direction of micro-patterns. Creating the protrusions 428 after the stent pattern has been laser cut may allow specific micro-patterns (pattern, height, length, width, angular orientation etc.) to specific locations. For example, a first strut 430a may include one or more protrusions 428a having a first angular orientation, a second strut 430b may include one or more protrusions 428b having a second angular orientation different form the first angular orientation, and a third strut 430c may include one or more protrusions 428c having a third angular orientation different from both the first and second angular orientations. This is just an example. It is contemplated that the protrusions 428 may be arranged such that two or more struts 430 have the same angular orientation.

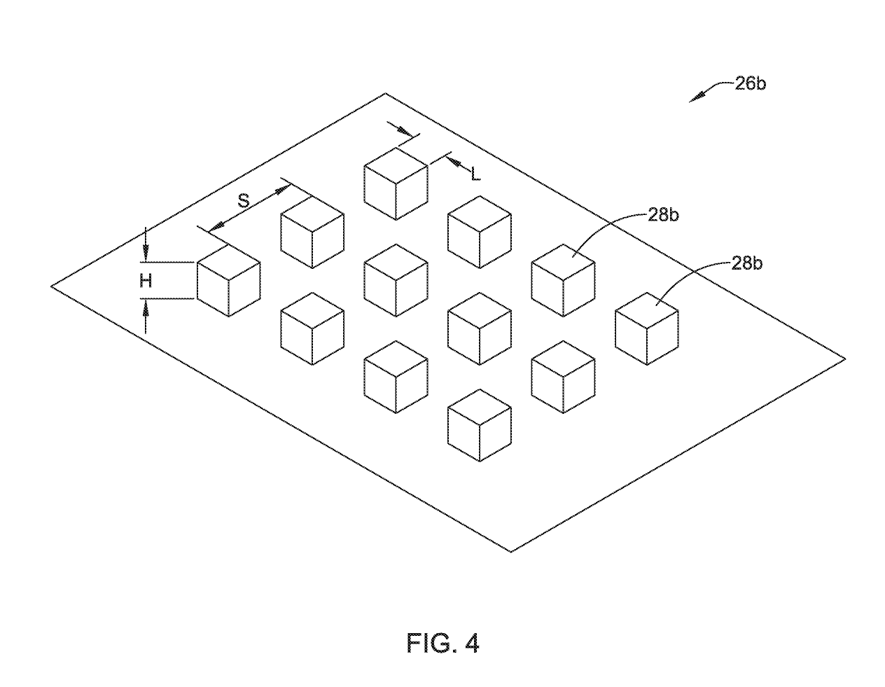

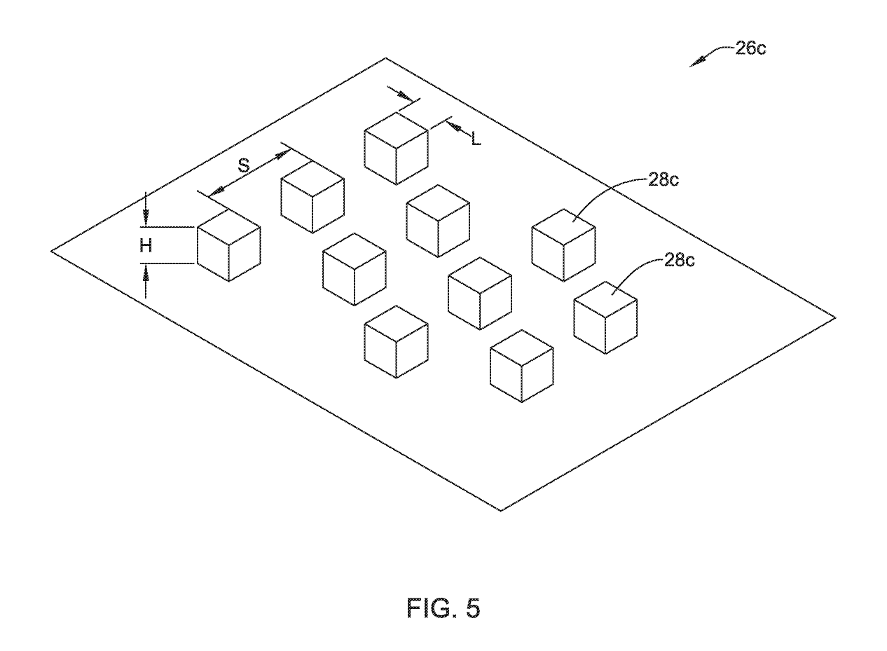

FIGS. 4 and 5 illustrate an enlarged and flattened view of illustrative protrusions 28b, 28c forming a micropatterned surface 26b, 26c. In FIGS. 4 and 5, the protrusions 28b, 28c have a square cross-sectional shape. Each protrusion 28b, 28c of the micropatterned surface 26b, 26c may have a first dimension, or length L, and a second dimension, or height H. The first dimension may be in the range of about 1 μm to 100 μm and the second dimension may be in the range of about 1 μm to 100 μm. It is contemplated that each protrusion 28b, 28c may be spaced apart from an adjacent protrusion by a distance S. The distance S may be selected based on the desired application. In some instances, the protrusions 28b may be uniformly distributed in an array, as shown in FIG. 4. In other instances, some protrusions 28c may be staggered from the protrusions 28c in adjacent rows to form a micropatterned surface 26c, as shown in FIG. 5. The protrusions 28b, 28c may be arranged in other patterns or configurations as desired, such as, but not limited to, helical rows or a random orientation. It is further contemplated that the angular orientation, pattern, height, length, width, and/or shape of the protrusions 28b, 28c can be varied, as desired, to achieve the desired effect.

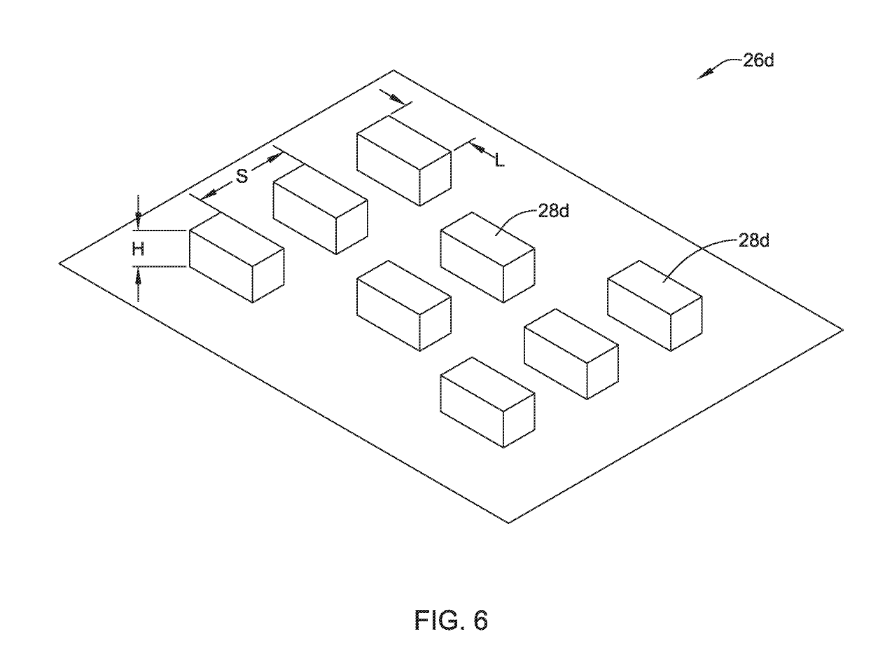

FIG. 6 illustrates an enlarged and flattened view of illustrative protrusions 28d forming a micropatterned surface 26d. In FIG. 6, the protrusions 28d have a rectangular cross-sectional shape. Each protrusion 28d of the micropatterned surface 26d may have a first dimension, or length L, and a second dimension, or height H. The first dimension may be in the range of about 1 μm to 100 μm and the second dimension may be in the range of about 1 μm to 100 μm. It is contemplated that each protrusion 28d may be spaced apart from an adjacent protrusion by a distance S. The distance S may be selected based on the desired application. In some instances, some protrusions 28d may be staggered from the protrusions 28d in adjacent rows to form a micropatterned surface 26d, as shown in FIG. 6. In other instances, the protrusions 28d may be uniformly distributed in an array. The protrusions 28d be arranged in other patterns or configurations as desired, such as, but not limited to, helical rows or a random orientation. It is further contemplated that the angular orientation, pattern, height, length, width, and/or shape of the protrusions 28d can be varied, as desired, to achieve the desired effect.

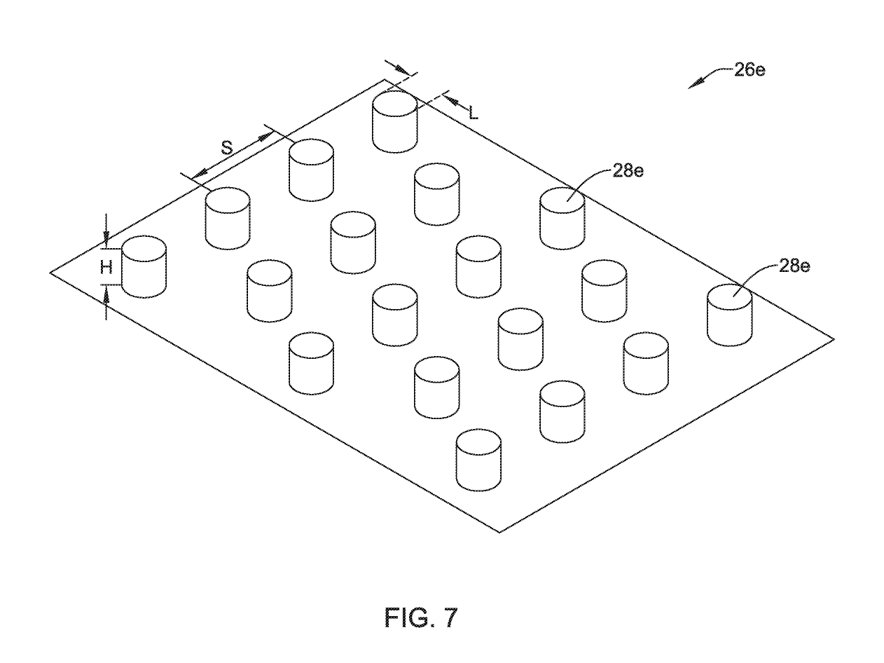

FIG. 7 illustrates an enlarged and flattened view of illustrative protrusions 28e forming a micropatterned surface 26e. In FIG. 7, the protrusions 28e have a circular cross-sectional shape. Each protrusion 28e of the micropatterned surface 26e may have a first dimension, or length L, and a second dimension, or height H. The first dimension may be in the range of about 1 μm to 100 μm and the second dimension may be in the range of about 1 μm to 100 μm. It is contemplated that each protrusion 28e may be spaced apart from an adjacent protrusion by a distance S. The distance S may be selected based on the desired application. In some instances, some protrusions 28e may be staggered from the protrusions 28e in adjacent rows to form a micropatterned surface 26e, as shown in FIG. 7. In other instances, the protrusions 28e may be uniformly distributed in an array. The protrusions 28e be arranged in other patterns or configurations as desired, such as, but not limited to, helical rows or a random orientation. It is further contemplated that the angular orientation, pattern, height, length, width, and/or shape of the protrusions 28e can be varied, as desired, to achieve the desired effect.

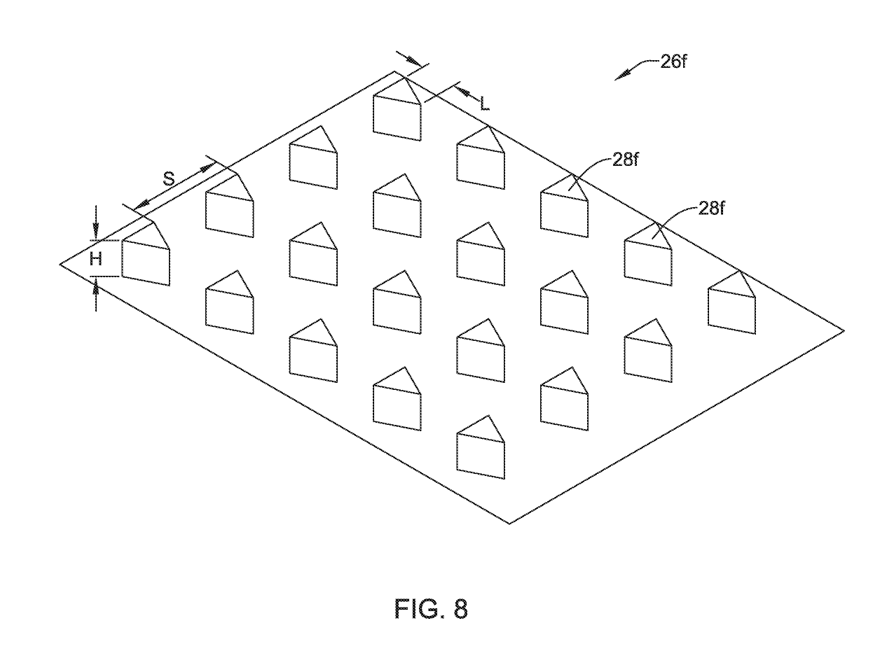

FIG. 8 illustrates an enlarged and flattened view of illustrative protrusions 28f forming a micropatterned surface 26f. In FIG. 8, the protrusions 28f have a triangular cross-sectional shape. Each protrusion 28f of the micropatterned surface 26f may have a first dimension, or length L, and a second dimension, or height H. The first dimension may be in the range of about 1 μm to 100 μm and the second dimension may be in the range of about 1 μm to 100 μm. It is contemplated that each protrusion 28f may be spaced apart from an adjacent protrusion by a distance S. The distance S may be selected based on the desired application. In some instances, the protrusions 28f may be uniformly distributed in an array, as shown in FIG. 8. In other instances, some protrusions 28f may be staggered from the protrusions 28f in adjacent rows to form a micropatterned surface 26f. The protrusions 28f may be arranged in other patterns or configurations as desired, such as, but not limited to, helical rows or a random orientation. It is further contemplated that the angular orientation, pattern, height, length, width, and/or shape of the protrusions 28f can be varied, as desired, to achieve the desired effect.

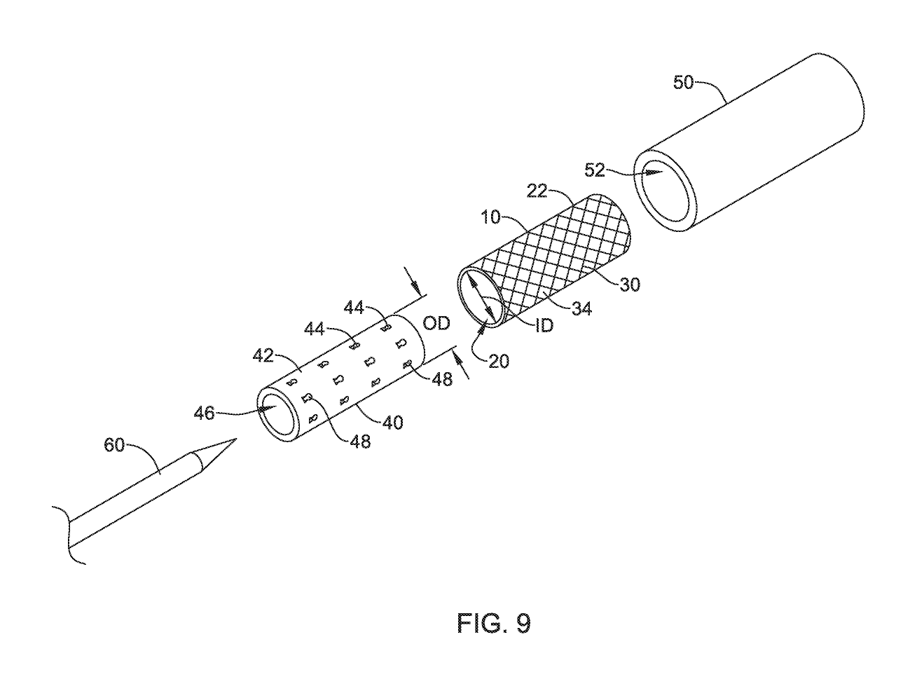

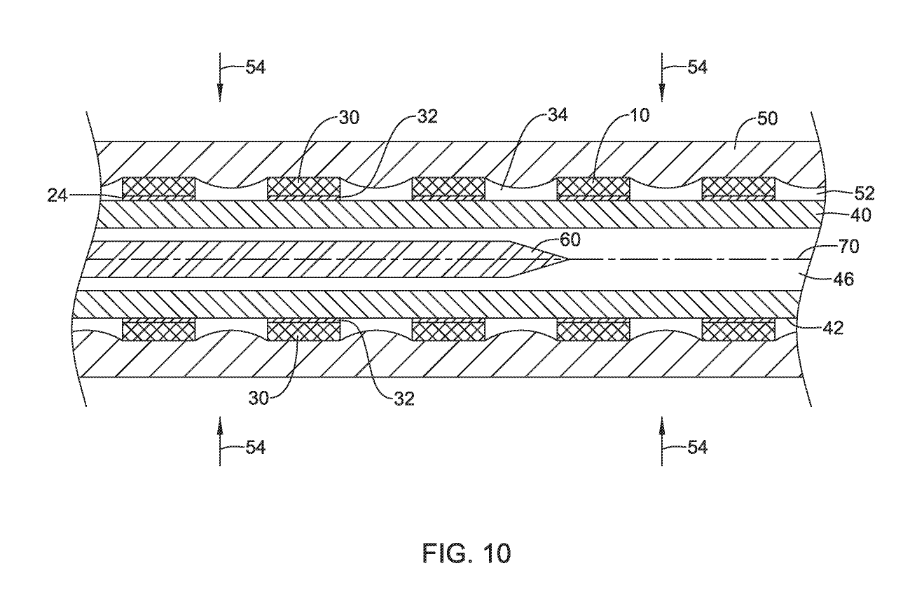

FIGS. 9 and 10 illustrate devices and methods for forming a micropatterned surface on an inside surface of a tubular element using a photo-etching process, or processes similar thereto. While the process is described relative to an endoluminal implant or stent 10, it is contemplated that the following method may be used to form a micropattern on the inner surface of any tubular device. It is further contemplated that the following method may be used on any concave surface of a curved surface by placing the outer surface 42 of the glass tube 40 against the concave surface. A glass tube 40 may include a negative pattern 44 of the desired micropattern coated on the outside surface 42 of the glass tube 40. The pattern 44 may include a number shapes 48 corresponding the negative images of the desired cross-sectional shape of the protrusions 28 to form the micropattern 26. The shapes 48 in the pattern 44 have been enlarged for clarity and are not to scale. In some embodiments, the negative pattern 44 may be formed using etches or inks as an optical mask. For example, the outer surface 42 of the glass tube 40 may be coated or prepared such that ultraviolet (UV) light passes through the shapes 48 forming the pattern 44 but is not allowed to pass through the remaining portion of the glass tube 40. The reverse configuration, in which UV light does not pass through the shapes 48 is also contemplated. The location of the optical mask (e.g. the negative pattern 44 or the remaining portion of the glass tube 40) will depend on the type of photoresist material used on the stent 10. While the tube 40 is described as glass, the tube 40 may be formed from any material that can be prepared to allow UV light to selectively pass through it.

The size, shape, and orientation of the shapes 48 of the negative pattern 44 may be selected based on the desired endothelial response once the stent 10 is placed in the body. The individual shapes 48 of the pattern 44 may have a length, the greatest distance between two opposite sides, in the range of about 1 μm to 100 μm. In some instances, the negative pattern 44 may be arranged as desired. For example, the pattern 44 may be uniformly distributed in an array, staggered, in helical rows, or a random orientation. These are just examples. In some embodiments, the pattern 44 may extend along the entire length of the glass tube 40. In other embodiments, the pattern 44 may extend over a portion of the outer surface 42 of the tube 40. In some instances, the pattern 44 may be formed in one or more discrete sections along a length or circumference of the outer surface 42 of the tube 40. For example, the pattern 44 may extend in intermittent rings around a perimeter of the tube 40 or in intermittent stripes along a length thereof. These are just examples. In some embodiments, the pattern 44 may be formed of all the same shape 48. In other embodiments, the pattern 44 may include two or more different shapes 48 depending on the desired response. For example, a portion of the pattern 44 may include shapes 48 which encourage cell growth, while another portion of the pattern 44 may include shapes 48 which inhibit cell growth.

The inner surface 24 (and/or outer surface 22) of the stent 10 may be coated or covered with a light sensitive photoresist layer 32 (FIG. 10). In some instances, the stent 10 may be dip coated or spray coated with the photoresist layer 32. It is contemplated that the photoresist layer 32 may be a positive resist in which the portion of the photoresist that is exposed to light is soluble to the photoresist developer or the photoresist layer 32 may be a negative resist in which the portion of the photoresist that is exposed to light becomes insoluble to the photoresist developer. Once the stent 10 has been coated with the photoresist layer 32, the glass tube 40 may be placed within the lumen 20 of the stent 10. The glass tube 40 may have an outer diameter OD that is approximately the same size as or slightly smaller than the inner diameter ID of the stent 10. For example, the outer diameter OD of the glass tube 40 may be sized to allow the glass tube 40 to freely slide within the lumen 20 of the stent 10 but also allow the inner surface 24, including the photoresist layer 32, of the stent 10 to be brought into contact with the outer surface 42 of the glass tube 40. It is contemplated that any remaining gap between the inner surface 24 of the stent 10 and the outer surface 42 of the glass tube 40 may be removed by pressing the stent 10 against the outer surface 42 of the glass tube 40. In some instances, the stent 10 and glass tube 40 assembly may be placed within the lumen 52 of a soft rubber tube 50, such as, but not limited to, a neoprene tube. The soft tube 50 may be used to apply a force in the direction of arrows 54 to press the stent 10 against the outer surface 42 of the glass tube 40. It is contemplated that other soft materials may be used in place of rubber or neoprene.

Once the inner surface 24 of the stent 10 is brought into contact with the outer surface 42 of the glass tube 40, one or more UV emitting fibers 60, or other UV light source, may be inserted into the lumen 46 of the glass tube 40. The UV fiber 60 may illuminate the inner surface 24 of the stent 10 with UV light through the negative pattern 44. In some instances, homogenous illumination may be obtained by rotating and/or translating the fiber(s) 60 about/along the longitudinal axis 70 during the illumination process. The UV fiber 60 may emit UV light along its entire length or from only a portion thereof.

Once the photoresist layer 32 has been exposed to the UV light through the negative pattern 44, the stent 10 may be etched and the photoresist layer 32 removed through conventional photo-etching procedures, resulting in a stent 10 having a micropatterned 26 inner surface 24 including a plurality of protrusions 28. The second dimension, or height H, of the protrusions 28 may be controlled by the processing times during the photo-etching process. While the UV light is described as penetrating the negative pattern 44, as discussed above, the UV light may penetrate the portion of the glass tube 40 surrounding shapes 48 of the pattern 44, as discussed above, and the stent 10 processed accordingly.

FIG. 10 illustrates a partial cross-section of the components of the photo-etching process during a portion of the photo-etching process. As can be seen in FIG. 10, the inner surface 24 of the stent 10 to be brought into contact with the outer surface 42 of the glass tube 40. It is contemplated that any remaining gap between the inner surface 24 of the stent 10 and the outer surface 42 of the glass tube 40 may be removed by pressing the stent 10 against the outer surface 42 of the glass tube 40. In some instances, a soft rubber tube 50, such as, but not limited to, a neoprene tube, may be used to apply a force to press the stent 10 against the outer surface 42 of the glass tube 40. In some instances, the tube 50 may fill, or partially fill, the gaps 34 between the struts 30 of the stent 10.

It is contemplated that a similar processing technique may be used to form a micropatterned surface 26 on the outer surface 22 of the stent 10. For example, the lumen 46 of the glass tube 40 may be sized to receive a stent 10 having a photoresist layer on the outer surface thereof. The stent 10 may be placed within the lumen 46 of the glass tube 40. The outer surface 22 of the stent 10 may be brought into contact with an inner surface of the glass tube 40. A UV light source may be provided to illuminate the stent 10 through the outer surface 42 of the glass tube 40. The outer surface 22 of the stent 10 may then be processed through conventional photo-etching techniques to create a plurality protrusions 28 on the outer surface 22 thereof.

In other embodiments, it may be desirable to form a micropatterned surface 26 on the inner surface 24 of a braided stent 10. However, the weaving of the filaments 30 may create an uneven inner surface. As such, the entire inner surface 24 of a braided stent 10 may not be capable of contacting the outer surface 42 of the glass tube 40. It is contemplated that the individual filaments 30 could be wound around the glass tube 40 and photo-etched in the manner described above. The filaments 30 may be processed in more than one batch such that the filaments 30 can be wound around the glass tube 40 in an orientation similar to the orientation the filament 30 will be in once the filament 30 is braided or woven into a stent 10. The individual filaments 30, including a micropatterned surface 26, may be braided or woven into a stent 10 having the micropatterned surface 26 of the filaments 30 along the inner surface 24 (or outer surface) of the stent 10, as desired.

In some embodiments, the photoresist layer 32 may be replaced with photocurable polymer material. In such an instance, the optical mask may be applied to the glass tube 40 such that UV light passes through the shapes 48 of the negative pattern 44. Once the stent 10 has been coated with the polymer material, the glass tube 40 may be placed within the lumen 20 of the stent 10. It is contemplated that any gap between the inner surface 24 of the stent 10 and the outer surface 42 of the glass tube 40 may be removed by pressing the stent 10 into and against the outer surface 42 of the glass tube 40. In some instances, a soft rubber tube 50, such as, but not limited to, a neoprene tube, may be used to apply a force to press the stent 10 against the outer surface 42 of the glass tube 40.

Once the inner surface 24 of the stent 10 is brought into contact with the outer surface 42 of the glass tube 40, one or more UV fibers 60, or other UV light source, may be inserted into the lumen 46 of the glass tube 40. The UV fiber 60 may illuminate the inner surface 24 of the stent 10 with UV light through the negative pattern 44. In some instances, homogenous illumination may be obtained by rotating and/or translating the fiber(s) 60 about and/or along the longitudinal axis 70 during the illumination process. The UV light may cause the polymer material adjacent to the pattern 44 to cross-link. The uncross-linked polymer may be washed away leaving the cross-linked polymer on the inner surface 24 of the stent 10. The cross-linked polymer may take the shape of the pattern 44 on the glass tube 40 resulting in a plurality of protrusions 28 forming a micropatterned surface 26. The height H of the protrusions 28 may be controlled by the thickness of the polymer material.

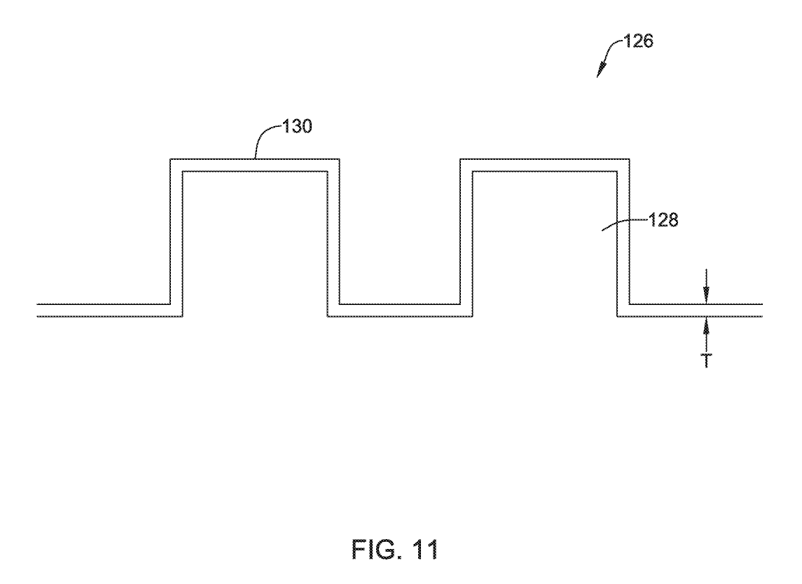

In some embodiments, it may be desirable to further process the implant to stimulate certain cell behavior. FIG. 11 illustrates a partial cross-section of another illustrative micropatterned surface 126 including a plurality of protrusions 128. The micropatterned surface 126 may be similar in form and function to the micropatterned surface 26 described above. Similarly, the micropatterned surface 126 may be formed in a similar manner to the micropatterned surface 26 described above. It is contemplated that specific ceramic coatings, such as but not limited to, titanium oxides (TiOₓ), may stimulate certain cell behavior. Atomic layer deposition (ALD) may be used to deposit a very thin layer of a coating 130 to the micropatterned surface. Such a process is described in, for example, in commonly assigned U.S. Pat. No. 8,864,816, entitled “Implantable medical devices incorporating x-ray mirrors”, which is incorporated herein by reference in its entirety. The coating 130 may be formed from a variety of ceramic, polymeric, as well as metal, ultrathin films. The coating 130 may have a thickness in the range of 1 to 50 nanometers (nm). In some instances, the coating 130 may cover the entire surface of the micropatterned surface 126 while in other instances, the coating may cover only a portion of the micropatterned surface 126.

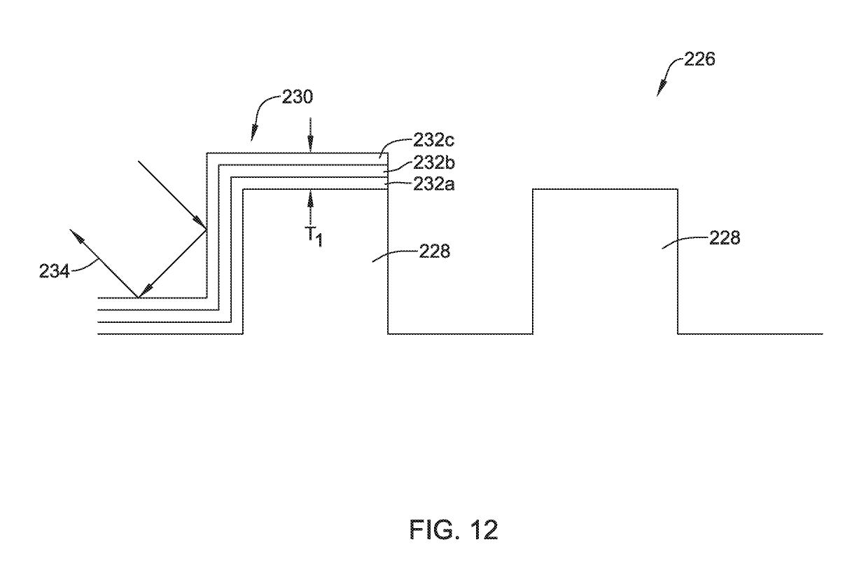

In some embodiments, it may be desirable to visualize an implant with x-rays once it has been placed in the body. FIG. 12 illustrates a partial cross-section of another illustrative micropatterned surface 226 including a plurality of protrusions 228. The micropatterned surface 226 may be similar in form and function to the micropatterned surface 26 described above. Similarly, the micropatterned surface 226 may be formed in a similar manner to the micropatterned surface 26 described above. It is further contemplated that ALD may be used to deposit an X-ray reflective nanolaminate coating 230 on the micropatterned surface 126. Nanolaminate coatings are described in, for example, in commonly assigned U.S. Pat. No. 8,864,816, entitled “Implantable medical devices incorporating x-ray mirrors”, which is incorporated herein by reference in its entirety The nanolaminate coating 230 may include a layered structure in which the individual layers 232a, 232b, 232c each have a thickness on the order of nanometers. While the coating 230 is illustrated as including three layers 232a, 232b, 232c, the coating 230 may have any number of layers desired. The total number of layers in the nanolaminate 230 may be chosen to optimize the reflectance angle (i.e. to produce detectable x-ray reflections at a variety of incident angles) and the intensity of the reflected signal 234. In some embodiments, the total number of layers is at least 8 or at least 10. In some embodiments, the total number of layers is greater than 1,000. The nanolaminate 230 may have a thickness T₁ in the range of 8 nm to 5 μm. In some instances, the coating 130 may cover the entire surface of the micropatterned surface 126 while in other instances, the coating may cover only a portion of the micropatterned surface 126.

It is further contemplated that it may be desirable to create different cell structures and/or behaviors at different parts of an implant. One solution may be to implant a very open porous structure in the body with all internal surfaces covered with a specific microtexture or micropattern, such as the micropatterned surfaces 26 described above, depending on the specific location in the 3-D implant. One way of making such a structure may be to use inverse-opal technology, whereby one assembles a bunch of microspheres (polymeric, wax, etc.), infuses this structure with a filler material or coating, and removes the microspheres by heating, etching, or chemical dissolution. What is left is the inverse opal structure. It is contemplated that the microspheres may be prepared first with an imprinted micropattern. In that case, one will get an inverse opal structure in which cells can be infused, but every internal surface is now patterned with a micro-pattern stimulating specific cell growth. Hence, if one takes different groups of microspheres and gives them specific different micropatterns and assembles these groups into a larger volume precisely placing types A, B, C, and D, for example, in different areas of the implant, one can create an implant with stimulates cell behavior based on the type and position in the implant. It is further contemplated that ALD may be used to cover the microspheres with a layer of additional material. Alternatively, or additionally, a metal film may be deposited on complex 3-D shapes utilizing super critical fluids.

The stents, implants, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. In some other embodiments, a superelastic alloy, for example a superelastic nitinol can be used to achieve desired properties.

In at least some embodiments, portions or all of the stents or implants may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or implants in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or implants to achieve the same result.

In some embodiments, a degree of Magnetic Resonance Imaging (MRI) compatibility is imparted into the stents or implants. For example, the stents implants s or portions thereof, may be made of a material that does not substantially distort the image and create substantial artifacts (i.e., gaps in the image). Certain ferromagnetic materials, for example, may not be suitable because they may create artifacts in an MRI image. The stents or implants or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for the stents or implants may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for manufacturing a curved medical device, the method comprising:
placing a glass tube having an optical mask on an outer surface thereof adjacent to a concave surface of a curved medical device, wherein the optical mask forms a pattern and the concave surface of the curved medical device having a light sensitive photoresist layer disposed thereon;
advancing an ultraviolet light source within a lumen of the glass tube;
illuminating the concave surface of the curved medical device with ultraviolet light through the glass tube, wherein the optical mask blocks ultraviolet light from passing through portions of the glass tube; and
etching the concave surface of the curved medical device to create a plurality of protrusions.

2. The method of claim 1, wherein the pattern is formed of a plurality of shapes, the shapes having a length in the range of 1 to 100 micrometers.

3. The method of claim 2, wherein the plurality of protrusion have a cross-sectional shape that is a negative image of the shapes of the pattern.

4. The method of claim 1, wherein illuminating the concave surface of the curved medical device with ultraviolet light through the glass tube comprises rotating and/or translating the ultraviolet light source along a longitudinal axis of the glass tube.

5. The method of claim 1, wherein placing a glass tube having an optical mask forming a pattern adjacent to a concave surface of a curved medical device further comprises applying a force to the curved medical device to bring the concave surface into contact with the outer surface of the glass tube.

6. The method of claim 1, wherein the pattern extends over a portion of the outer surface of the glass tube.

7. The method of claim 1, wherein the pattern extends over the entire outer surface of the glass tube.

8. The method of claim 1, wherein a height of the plurality of protrusions is determined by a processing time of the etching of the concave surface.

9. A method for manufacturing a tubular medical device, the method comprising:
placing a glass tube having an optical mask on an outer surface thereof within a lumen of a tubular medical device, wherein the optical mask forms a pattern of shapes;
advancing an ultraviolet light source within a lumen of the glass tube;
illuminating an inner surface including a photoresist coating of the tubular medical device with ultraviolet light through the glass tube, wherein the optical mask blocks ultraviolet light from passing through portions of the glass tube; and
etching the inner surface of the tubular medical device to create a plurality of protrusions.

10. The method of claim 9, further comprising applying a force to an outer surface of the tubular medical device to bring the inner surface of the tubular medical device into contact with the outer surface of the glass tube prior to illuminating the inner surface of the tubular medical device.

11. The method of claim 9, wherein the shapes in the pattern of shapes having a length in the range of 1 to 100 micrometers.

12. The method of claim 9, wherein illuminating an inner surface of the tubular medical device comprises rotating and/or translating the ultraviolet light source along a longitudinal axis of the glass tube.

13. The method of claim 9, further comprising applying a coating over the plurality of protrusions.

14. An endoluminal implant comprising:
an elongated tubular body having an inner surface and an outer surface;
wherein the inner surface comprises a micropatterned surface including a plurality of protrusions, the plurality of protrusions formed as a monolithic structure with elongated tubular body.

15. The endoluminal implant of claim 14, wherein the plurality of protrusions each have a length in the range of 1 to 100 micrometers.

16. The endoluminal implant of claim 14, wherein the plurality of protrusions each have a height in the range of 1 to 100 micrometers.

17. The endoluminal implant of claim 14, wherein the micropatterned surface extends over a portion of the inner surface of the elongated tubular body.

18. The endoluminal implant of claim 14, wherein the micropatterned surface extends over the entire inner surface of the elongated tubular body.

19. The endoluminal implant of claim 14, further comprising a coating disposed over the micropatterned surface, the coating selected to stimulate cell growth.

20. The endoluminal implant of claim 14, wherein the plurality of protrusions each have cross-sectional shape selected to promote cell growth.
